(19) United States
(12) Patent Application Publication
(10) Pub. No.: US 2022/0076790 A1
(43) Pub. Date: Mar. 10, 2022

(54) INCORPORATING VARIANT INFORMATION INTO OMICS

(71) Applicant: Mahalakshmi N.A. SRINIVASAN, Mumbai (IN)

(72) Inventor: Mahalakshmi N.A. SRINIVASAN, Mumbai (IN)

(73) Assignee: NAS BIOVENTURES LLC, Newark, DE (US)

(21) Appl. No.: 17/419,286

(22) PCT Filed: Nov. 18, 2019
(86) PCT No.: PCT/IN2019/050851

§ 371 Date: Jun. 28, 2021

(30) Foreign Application Priority Data
Dec. 27, 2018 (IN) 201821049263

(51) Int. Cl. G16B 50/20; G16B 20/20; G16B 30/20

(52) U.S. Cl. CPC G16B 50/20; G16B 30/20; G16B 20/20

(57) ABSTRACT

The invention is a code to incorporate variant information into omics data. The omics data so obtained could be used for determining the biomolecules, pathways, deciphering the taxonomy, probe designing, understanding evolutionary transitions at the omics level, data analysis, annotation, interpretation, visualization and any further studies. The omics data obtained after incorporation of variants is in compliance with the file formats of omics data and can be used as any other omics data in Bioinformatics software(s) or as standalone tool. The code could be hosted as an online tool. The code has been originally developed using R. The invention involves approaches to enable editing of omics data, using code which transforms the data type, manipulates, incorporate information in a sequential manner and converts them back to the same omics file format specification. The code could be written using various programming languages and compatible with all operating systems.

### TECHNICAL FIELD

The invention belongs to the field of Bioinformatics in order to incorporate variant information into Omics data.

### BACKGROUND

In accordance with the Census of Marine Life and as featured in Science Daily, there are about 8.7 million (give or take 1.3 million) estimated new species on Earth; the most precise calculation ever offered, with 6.5 million species on land and 2.2 million in oceans.

With Natural Selection which is the central mechanism of evolutionary changes in species, an array of phenotypic and genotypic variations can be observed in nature. It is natural selection which results in organisms more likely to survive in a given ecosystem and reproduce and thus lead to speciation. Speciation is defined as the formation of new and distinct species in the course of evolution.

Accordingly, the study of the omics data, for example Whole Genome Sequences of these species is vital. Only comparison with sequences reported in our repository doesn't suffice and having the entire sequence to understand better the genotype, phenotype and kind of biomolecules produced is required, especially when the percentage of similarity with data in repository is 70% to 85%.

While dealing with New Species we would come across situations when we have to decide about the taxonomy of these species. This requires thorough investigation of the biomolecules, pathways of the species.

In the pursuit to study these new/distinct species, the omics data of these species are required which could then be used to design probes. There are numerous technologies and advancements done in the kind of probes designed to analyze a given species.

Studying new/distinct species due to speciation is vital to better understand how they place themselves in the ecosystem or environment. This is especially important while dealing with clinical samples, pathogens, environmental samples. Speciation has got to do with the environment and interactions between life forms. For example, in the case of pathogens, the speciation has got to do a lot with host-pathogen interactions.

Variants includes Insertion, Deletion, Single Nucleotide Polymorphism (SNP)/Single Nucleotide Variation (SNV), MNV (Multiple Nucleotide Variants), and Replacement.

Current technologies enables us to identify biological specimen based on comparison to known species sequence information existent in the databases/repositories. This inference that the biological specimen under study is a variant of a known species is based on the percentage of similarity obtained using the algorithms to find best match in the databases or repositories.

There are situations where the percentage of similarity/identity obtained during alignment is high but the coverage is low, in other words the percentage of identity is referring to only the regions which were compared between two or more sequences and when low coverage, the extent of uncompared regions between the sequences is high. In such a situation, considering one is working with an environmental isolate, it would become difficult to decipher the taxonomic identity of the microbe. If we have a tool which could incorporate these variants instantaneously into the omics data, as part of analysis, then it would be more productive and enable the research to progress faster, with correct insights at the biomolecular level about the species being studied.

This Bioinformatics tool could be used to understand the evolutionary transitions happening at the omics level.

This invention is a code by which sequence file(s) in various formats such as fasta files could be edited to incorporate variant information. For instance, we have the best match information found from the repositories and the data of how the species being investigated varies from it. This code enables us to incorporate the variants data into the omics file and obtain the complete sequence of the species, for example Whole Genome Sequence. Following this, the taxonomy could be deciphered based on thorough investigation of the biomolecules, and further analysis done. The complete sequence thus obtained helps in probe designing. We obtain huge amount of data on a daily basis with Next Generation Sequencing. Many are environmental isolates and for identifying them with the existent approaches, we use the numerous bioinformatics softwares and tools. However, with low coverage while comparing with existing sequence in the databases, it is highly possible that what is thought to be a variant, could even be a new species or an effect of speciation.

There are situations where multiple species turn out to be the best match from the database or repositories, with minor differences in percentage of similarities. In such situations, incorporating the variant information and then studying the complete sequence will enable understand the array of biomolecules produced, the pathways of the species being investigated and the intricate differences with regard to biomolecules and pathways.

With this invention, the complete sequence of the species being investigated could be obtained and then used for appropriate taxonomic identification, probe designing, understanding the biomolecular processes and pathways, understanding the evolutionary transitions at the omics level and further data analysis using omics data.

Further, it is essential to note the pitfalls associated with 16S rRNA identifications, that can be addressed using this invention. “Although it has been demonstrated that 16S rRNA gene sequence data on an individual strain with a nearest neighbor exhibiting a similarity score of <97% represents a new species, the meaning of similarity scores of >97% is not as clear. This latter value can represent a new species or, alternatively, indicate clustering within a previously defined taxon. DNA-DNA hybridization studies have traditionally been required to provide definitive answers for such questions. Whereas 16S rRNA gene sequence data can be used for a multiplicity of purposes, unlike DNA hybridization (>70% reassociation) there are no defined “threshold values” (e.g., 98.5% similarity) above which there is universal agreement of what constitutes definitive and conclusive identification to the rank of species”. “The explosion in the number of recognized taxa is directly attributable to the ease in performance of 16S rRNA gene sequencing studies as opposed to the more cumbersome manipulations involving DNA-DNA hybridization investigations. DNA-DNA hybridization is unequivocally the “gold standard” for proposed new species and for the definitive assignment of a strain with ambiguous properties to the correct taxonomic unit”.

Ref: 16S rRNA Gene Sequencing for Bacterial Identification in the Diagnostic Laboratory: Pluses, Perils, and Pitfalls. J. Michael Jando and Sharon L. Abbott. J Clin Microbiol. 2007 September; 45(9): 2761-2764.PMCID: PMC2045242, PMID: 17626177

Another reference indicative of disadvantages of 16S rRNA based phylogenetic studies is as follows: “Although the 16S rRNA method has served as a powerful tool for finding phylogenetic relationships among bacteria, because of its molecular clock properties and the large database for sequence comparison, the molecule is too conserved to provide good resolution at the species and subspecies levels”.

Ref: Bacterial Species Determination from DNA-DNA Hybridization by Using Genome Fragments and DNA Microarrays. Jae-Chang Cho, James M. Tiedje. Applied and Environmental Microbiology. DOI: 10.1128/AEM.67.8.3677-3682.2001

This invention helps in obtaining the omics data of the species being investigated by incorporating variant information, which could then be useful in all the further experimentation and inferences to be drawn.

### OBJECTIVES OF THE INVENTION

The invention enables to incorporate variant information into the omics data. This shall be useful in obtaining the complete sequence of the species being studied, understand the complete biomolecular profiles and pathways of the species, decipher the correct taxonomic designation, help in probe designing for these species and understanding the evolutionary transitions at the omics level.

### SUMMARY

The invention is a tool to incorporate variant information into a given omics data, to use the complete sequence for further analysis, interpretations. These could be used for new/distinct species, especially useful while studying pathogens, clinical samples and environmental samples. However, this tool could also be used for samples with minor variations from the known species.

In accordance with the Census of Marine Life and as featured in Science Daily, there are about 8.7 million (give or take 1.3 million) estimated new species on Earth; the most precise calculation ever offered, with 6.5 million species on land and 2.2 million in oceans. With Natural Selection which is the central mechanism of evolutionary changes in species, an array of phenotypic and genotypic variations can be observed in nature. It is natural selection which results in organisms more likely to survive in a given ecosystem and reproduce and thus lead to speciation. Speciation is defined as the formation of new and distinct species in the course of evolution.

The invention enables to incorporate variant information into the omics data. This shall be useful in obtaining the complete sequence of the species being studied, understand the complete biomolecular profiles and pathways of the species, decipher the correct taxonomic designation and help in probe designing, understanding the evolutionary transitions at the omics level, further data analysis, annotation for these species. Taxonomic studies are mainly based on 16S rRNA which have their own set of pitfalls and valuable information may be lost.

The code could be integrated into the existing software workflows or could be used independently as a standalone code—downloadable or as online tool.

The code can be implemented using any of the programming languages and compatible with all operating systems.

The code focuses on manipulating the data type and incorporating variant information.

### BRIEF DESCRIPTION OF THE DRAWINGS

FIG. 1 illustrates the steps followed to incorporate the variants into a omics data file of the species being studied.

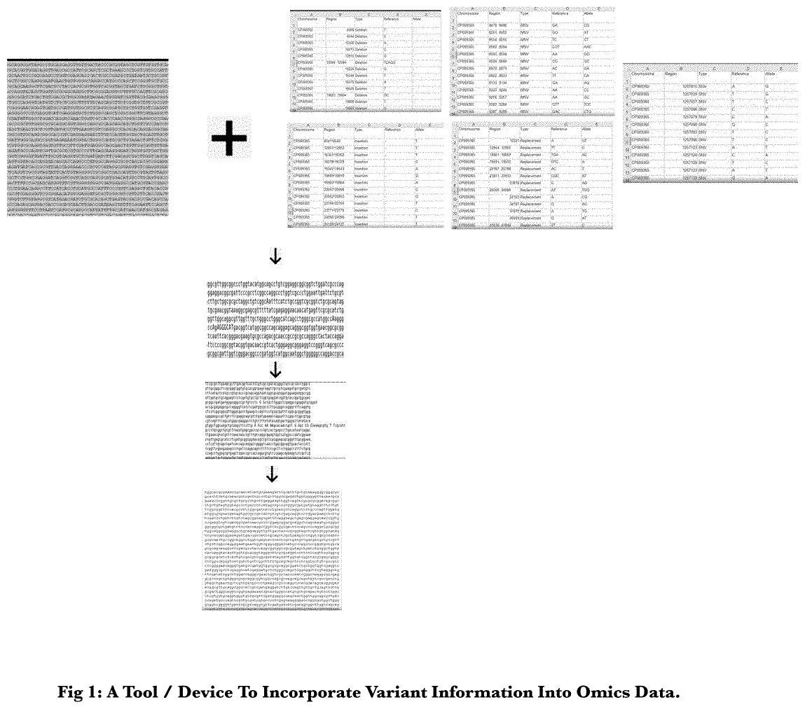

### DETAILED DESCRIPTION OF THE INVENTION

In this detailed description, one of the applications of the code is being described in detail.

There were 18 bacterial isolates obtained from the environment. The Whole Genome sequencing of the 3 isolates were done using Next Generation Sequencer and further analysis using the Bioinformatics software. Biochemical tests were done manually and using two automated biochemical test systems. Morphological characteristics and colony characteristics were studied.

The observations were as follows:

Manual biochemical tests results couldn't help conclude about the taxonomy of these environmental species. One of automated system used reported the isolate to be Pseudomonas fluorescence/Pseudomonas putida while the other automated system reported the isolate to be Chromobacterium violaceum. There were literatures about some of the bacterium placed under Chromobacterium that need to be placed under Pseudomonas genus.

At the software level, numerous commercial and free Bioinformatics software were used. The best match reported was of Pseudomonas species, with low coverage and high percentage identity. There were huge amount of variants reported in comparison to the best match species found, the similarity to the known species in the repositories was found to be in the range of 75%-85%.

With all this, the best approach left was to first get the whole genome sequence incorporated with the variant information, which could then be used for further studies.

With all the variants incorporated into the omics file, this file was then once again used to check if the best sequence match can be found in the repositories such as that of NCBI BLAST. However, a best match with high percentage of identity and high coverage could not be obtained for these 3 bacteria, indicating that they were new species.

Please Note: The invention isn't restricted to this one mentioned case study or application. The invention gives one the ability to obtain the complete sequence of the species being studied, to understand the complete biomolecular profiles and pathways of the species, decipher the correct taxonomic designation, help in probe designing, understanding the environmental transitions at the omics level and further data analysis, annotation for these species.

The steps involved in incorporating the variant information into the omics data involves:
Loading the omics data into the software, the code has been developed using the Data Science tool—R
The data type of this file is then converted to a form which makes it feasible to make editing at the desired positions in the lengthy sequence. Here, editing means ability make insertion(s), deletion(s), substitution(s) and enable replacement(s) in the sequence.
Once, the data is of a data type which could be edited to incorporate variations at the correct location, then all the variants could be incorporated sequentially at the correct specified location, as indicated in the input variant file.
After the variant information are incorporated, steps are implemented to change the data type such that we could obtain the same file format, which was originally transformed to incorporate variants.
This data so obtained with variant information incorporated could be imported, exported analyzed and used for further studies.

### ADVANTAGES OF THE INVENTION

The invention enables:
Obtaining the complete sequence of the species being studied, following incorporation of variants.
Probe designing,
Understanding pathways of the species,
Place species under the correct taxonomic designation,
Understanding evolutionary transitions at the omics level,

This invention is particularly useful in analyzing the environmental samples, pathogenic samples, clinical samples. With Natural Selection which is the central mechanism of evolutionary changes in species, an array of phenotypic and genotypic variations can be observed in nature. It is natural selection which results in organisms more likely to survive in a given ecosystem and reproduce and thus lead to speciation. Speciation is defined as the formation of new and distinct species in the course of evolution. The invention addresses in obtaining the complete sequence of species.

The code could be written using any of the programming languages, could be used as a standalone tool or included in the existent Bioinformatics software. The code could be hosted as an online tool.

1. A code which enables editing of omics data (a data type) to incorporate variant information and then transforms the data type back to the original file format, characterized by:
Obtaining omics data with variants incorporated,
The omics data obtained after incorporation of variants is in compliance with the file formats of Omics data and can be used as any other Omics data obtained from technologies such as Next Generation Sequencing and existent Bioinformatics software(s), tool(s).
Using the omics data with variant incorporated for determining the biomolecules, pathways of the species,
Using the omics data with variant incorporated for deciphering the taxonomy,
Using the omics data with variant incorporated for probe designing,
Using the omics data with variant incorporated for understanding the evolutionary transitions,
Using the omics data with variant incorporated for data analysis, annotation, interpretation and visualization or any further studies, using bioinformatics software(s).
Using the omics data with variant incorporated in studying clinical samples, pathogens, environmental samples.
Using the omics data with variant incorporated in studying the variants of known organisms.

2. A method of claim 1, which was originally developed using R and can be a standalone code or an online tool or integrated into existent bioinformatics software.

3. A method of claim 1, wherein the code could be developed using any programming language and compatible with all operating systems.

4. A method of claim 1 which enables transformation of omics data to a data type which can be edited at specified locations, as indicated in the user input file of variants.

5. A method of claim 1 which enables sequential incorporation of variants into the omic data file, such that the variant gets incorporated at the correct specified location in the input file.

6. A method of claim 1 where the variant incorporation could mean addition/insertion, deletion, substitution and replacement of data.

7. A method of claim 1 whereby the program transforms the data type to a form which can then be used to obtain the original file format of the omics data.

8. The method of claim 1 whereby the omics data obtained on incorporating the variant information is available for all processes such as importing, exporting, analysis, annotation using the various free and commercial bioinformatics software(s).

9. The method of claim 1 which is fast, not error prone

10. A method of claim 1, whereby the incorporation of variants is a sequential process, could be done batch wise, using multiple variant files as input or a single file with all variant information. The transformation from data type to omic data file format could happen during any stage of variant incorporation and not necessarily after all the variant information has been incorporated.

11. The system of claim 1 wherein the code could be provided as a packaged tool/software.
